(19) United States
(12) Patent Application Publication
(10) Pub. No.: US 2010/0203576 A1
(43) Pub. Date: Aug. 12, 2010

(54) PROCESS FOR DETERMINATION OF MICROORGANISMS' RESISTANCE TO ANTIBIOTICS

(75) Inventor: Christian CUREL, Laverune (FR)

Correspondence Address: YOUNG & THOMPSON, 209 Madison Street, Suite 500, Alexandria, VA, 22314, US

(73) Assignee: INTELLIGENCE ARTIFICIELLE APPLICATIONS, Perols (FR)

(21) Appl. No.: 12/700,105

(22) Filed: Feb. 4, 2010

(30) Foreign Application Priority Data
Feb. 9, 2009 (FR) 09 50782

(51) Int. Cl. C12Q 1/02

(52) U.S. Cl. 435/29

(57) ABSTRACT

A process for determination of microorganisms' projected resistance to antibiotics that includes analyzing a first microorganism by mass spectrometry so as to obtain a first mass spectrum of the first microorganism, whereby the process is characterized in that an analytical device carries out the following stages:
(i) Comparing the first mass spectrum to mass spectra that are contained in a data base, so as to obtain a list of one or more microorganisms that are identical or at least similar to the first microorganism, and whose resistance to given antibiotics is known,
(ii) Implementing a projected analysis of the first microorganism's resistance to antibiotics based on the resistance to antibiotics of the microorganism or microorganisms that are identical or at least similar, obtained in stage (ii).

This invention relates to a process for quick determination of microorganisms' projected resistance or sensitivity to antibiotics.

When an infection is suspected in an individual that may be a patient or an animal, a sample is sent to be analyzed in a laboratory. When the infection is assumed to be bacterial, two operations have to be executed. First of all, the first operation consists in identifying, in the sample, the microorganism in question: type of bacteria, yeasts, or fungi. Then, the second operation consists in executing a test of the identified microorganism's sensitivity to antibiotics. The testing of several antibiotics with regard to an organism is called the antibiogram.

The methods that have been used to date for executing these two stages require a length of time of several days, and even several weeks, to obtain these results.

The first stage consists in cultivating the microorganism. This stage generally takes one day for common organisms, but can spread over several days (fungi, yeasts), and even weeks (for example for mycobacteria).

It is carried out by inoculation of the sample on one or more culture media, or else by use of automatic devices in predefined cases, such as hemocultures (culture of blood samples), or mycobacteria research, for example.

When the cultivation is done, chemical tests generally make it possible to define the type of bacteria. The identification of the microorganisms is then launched from isolated colonies; in general, the antibiogram is launched simultaneously.

The antibiogram consists in determining the sensitivity of the antibiotics (or antifungal agents) with regard to the isolated microorganism. Its object is to determine, for each antibiotic (or antifungal agent), the smallest growth of antibiotic (antifungal agent) that makes it possible to inhibit the growth of the microorganism: this is the minimum inhibiting concentration or cmi. Several antibiotics (antifungal agents) are tested with regard to a microorganism.

The identification consists in finding the name of the microorganism that is responsible for the infection.

Several methods are used.

During the method of biochemical identification, several tests are carried out with regard to the microorganism. The combination of these tests provides a probability of the presence of a certain microorganism; the result is obtained by statistical comparison of the results with a pre-established data base of microorganisms that is documented with regard to the tests that are carried out. This is the method that is used universally.

For several years now, the appearance of chromogenic culture media (media supplemented with substrates) has made it possible to cultivate organisms, but has also allowed the presumed identification of certain targeted organisms (change of color of the colonies in the presence of a certain organism); this method, widely used in certain countries, targets only a very limited number of microorganisms and requires more or less frequently a confirmation or additional tests.

In targeted cases, generally for important pathologies (septicemias, . . . ), it is possible to use molecular biology methods (in general, DNA analysis); these methods have several drawbacks: they target only several organisms (it is necessary to develop a minimum initiation by type of organism), they are heavy enough to use (infrastructure constraints, specialized personnel), they are very cumbersome, and the length of time for obtaining results remains several hours.

Finally, a new technology for identification of the microorganisms has been introduced: the identification is done by MALDI-TOF technology (flight and desorption time/matrix-assisted laser ionization mass spectrometry). The identification rests on the analysis of the molecular weights of the samples. The MALDI principle is to carry out a “mild” ionization of the microorganism so as to keep the large proteins. The organism is extracted from a culture medium, a suspension or a sample; it is deposited on a plate with a given position; a matrix solution is added. The position where the organism is located is bombarded by laser fire; the molecules are then ionized and then accelerated via a magnetic field: they are separated according to their mass-to-charge ratio. The separated ions are detected, and this signal is sent to a data system where the mass-to-charge ratios are stored as well as their relative abundance; the system then constitutes a mass spectrum of the organism that is to be analyzed. The latter is processed by software and compared to a data base of spectra corresponding to identified organisms. The name of the analyzed organism is thus obtained.

Regardless of the method that is used for the production of the antibiogram, a significant length of time is required to obtain the results: at least several hours (5 hours minimum).

In the extreme majority, the results are available in the morning following the start-up date (because, even if the results are available 8 hours or 10 hours after start-up, there is no one to validate them, and, moreover, there is no one to receive them on the medical side). For some microorganisms, several days are necessary, and even several weeks (mycobacteria).

In addition, in a large number of cases, additional tests have to be executed to confirm the suspected resistance mechanisms. These additional tests generally take one day.

Taking into account the length of time for obtaining results from the antibiogram, the therapeutic treatment is always undertaken before having the results from the laboratory. In general, a sample is taken, and treatment is undertaken immediately afterwards. The treatment that is undertaken is defined empirically from various criteria: clinical signs that provide a presumption of pathology; state of the patient (immunodepressed, allergies, . . . ); established protocols (consensus conferences); local habits, etc.

In general, whereby the type of bacteria and the results of the antibiogram are not known, broad-spectrum antibiotics (covering a multitude of cases) are prescribed. This has the advantage of generally protecting the patient promptly, but it involves major drawbacks:
Broad-spectrum antibiotics generally have significant secondary effects for the patient;
The prescription of broad-spectrum antibiotics develops the resistance of the bacteria with regard to these antibiotics or with regard to other classes of antibiotics; this is the most acute problem because it brings about certain therapeutic impasses for bacteria that become multi-resistant (resistant to several classes of antibiotics);
Finally, the prescription of broad-spectrum antibiotics has a significant economic impact because the treatments are generally expensive.

The treatment is generally re-evaluated at two or three days, when the clinician has the antibiogram results. The re-evaluation is performed based on results received from the laboratory and based on the changes in the patient's condition.

The document US 2008/0009029 describes a process that consists in:
Cultivating a preferably previously identified microbe with a given antibiotic in a culture medium and optionally establishing a common sample,
Then comparing the mass spectrum that is obtained with a data base that already comprises numerous microorganism spectra that are resistant or non-resistant to specified antibiotics.

The “Intact Cell Mass Spectrometry Used to Type Methicillin-Resistant Staphylococcus aureus: Media Effects and Inter-Laboratory Reproducibility” publication by J. Walker et al. relates to the effects of the use of different culture media or different spectrometric equipment so as to study the reproducibility of the MALDI-TOF technique for identifying the methicillin-resistant Staphylococcus aureus.

Likewise, the “Rapid Discrimination between Methicillin-Sensitive and Methicillin-Resistant Staphylocossus aureus by Intact Cell Mass Spectrometry” publication by Valerie Edwards-Jones describes the use of the MALDI-TOF technique for identifying the Staphylococcus aureus species that is sensitive to methicillin or resistant to methicillin.

The rapid information of the clinicians on microorganisms' sensitivity and resistance is therefore a major objective.

The object of this invention is a process for determination of microorganisms' projected resistance or sensitivity to antibiotics that prevents all or part of the above-mentioned drawbacks.

Microorganism is defined as any microscopic living organisms, such as bacteria, yeast, protozoa, viruses, archaea, etc., which may or may not be pathogenic.

Also, “resistance or sensitivity to antibiotics” is defined as the capacity of a microorganism to withstand the effects of antibiotics. The more resistant or insensitive a microorganism will be to an antibiotic, the less suitable this antibiotic will be for the treatment of the infection or illness produced by this microorganism.

For this purpose, the object of the invention is a process for determination of microorganisms' projected resistance to antibiotics that consists in analyzing a first microorganism by mass spectrometry so as to obtain a first mass spectrum of said first microorganism, whereby said process is characterized in that an analytical device carries out the following stages:
(i) Comparing said first mass spectrum to mass spectra that are contained in a data base, so as to obtain a list of one or more microorganisms that are identical or at least similar to said first microorganism, and whose resistance to given antibiotics is known,
(ii) Implementing a projected analysis of said first microorganism's resistance to antibiotics based on the resistance to antibiotics of the microorganism or microorganisms that are identical or at least similar, obtained in stage (ii).

The invention therefore makes it possible to inform the clinician as early as possible of the risk of resistance of certain antibiotics (antifungal agents) with regard to the microorganisms. This makes it possible to adapt a therapeutic treatment very quickly by modifying it either because it leads to a failure or because it is poorly adapted and can bring about unfortunate consequences for the patient or the environment (broad-spectrum antibiotics).

Advantageously, the mass spectrometry is a flight and desorption time/matrix-assisted laser ionization mass spectrometry (MALDI-TOF).

Preferably, the data base is a base of spectra that is local to a laboratory and/or a base of pre-defined spectra, in which the resistance of the organisms associated with these spectra is known.

Advantageously, the analytical device comprises a computer and software.

The invention will be better understood, and other objects, details, characteristics and advantages of the latter will appear more clearly, during the following description of a particular embodiment of the invention, provided only by way of illustrative and nonlimiting example.

A urine specimen from a patient X has been analyzed according to stage (i) by a mass spectrometer. In particular, the spectrometer was a MALDI-TOF, i.e., a flight and desorption time/matrix-assisted laser ionization mass spectrometer.

Following this analysis, it appeared that this sample contained Escherichia Coli. A mass spectrum of this microorganism also has been produced and obtained by the MALDI-TOF spectrometer.

Using an analytical device that comprises, among other things, a computer and software, this Escherichia coli mass spectrum has been compared with a list of spectra that are similar to other Escherichia coli strains. This list of similar spectra is stored in advance in a data base that is present in the software.

This data base also comprises the resistance of the microorganisms combined with this list of spectra, i.e., the resistance of specific microorganisms was already analyzed by an antibiogram, for example, and was recorded in the data base.

The analytical device then defines a limit of the similarity (in % or by threshold) of the Escherichia coli spectrum with the spectra that are contained in the data base, for example, the spectra of other Escherichia coli strains.

In the example of the urine specimen, the analytical device provides very close strains that are listed in Table I below:

TABLE I

| | Species | Type | Note | ID. of Spectrum | Score |
|---|---|---|---|---|---|
| 1 | Escherichia coli | Escherichia | Blood Agar | TYYYOTPPORRE | 98.5 |
| 2 | Escherichia coli | Escherichia | Blood Agar | TVVVYSQAEGG | 97.8 |
| 3 | Escherichia coli | Escherichia | Blood Agar | TVVFGSGFDRGG | 84.2 |
| 4 | Escherichia coli | Escherichia | Blood Agar | TYYYOTFDRRRE | 83.6 |

Then, the analytical device calculates the resistance of similar strains that are found and establishes a ratio (as in Table II) of projected resistance. This ratio can comprise only resistance and/or indicate sensitivity.

The projected results are calculated statistically from the results of similar strains. It is possible to use different statistical tests (average, etc.).

TABLE II

| | MO⁽¹⁾ LIST IN | | | | |
|---|---|---|---|---|---|
| TESTED | DATA BASE | | | | PROJECTED |
| ANTIBIOTICS | 1 | 2 | 3 | 4 | RESISTANCE |
| SIMILARITY | 98.5% | 97.8% | 84.2% | 83.6% | / |
| AMOXICILLIN | R | R | R | R | 100% (12) |
| AMOXI + CLAVU | I | I | R | I | 100% (12) |
| PIPERACILLIN | R | R | R | R | 100% (11) |
| CEPHALOTIN | R | R | R | R | 100% (12) |
| CETRIAXONE | S | S | S | S | 0% (12) |
| GENTAMICIN | S | S | S | S | 0% (12) |
| CIPROFLOXACIN | R | R | NI | I | 100% (11) |
| TRIMETHO + SULFA | S | S | S | S | 0% (12) |

⁽¹⁾MO: Microorganism

In Table II above, “R” means “significant resistance,” “I” means “average resistance,” “S” means “without resistance,” and “NI” corresponds to “not indicated.”

Thus, it can be seen very quickly that the Escherichia coli species 1 to 4, similar—between 98.5 to 83.6%—to the Escherichia coli of the analyzed urine specimen, are resistant to amoxicillin, piperacillin, and cefalotin, while they are not resistant to cetriaxone, to gentamicin, and to the trimethoprime-sulfamethoxazole mixture. Consequently, the Escherichia coli strain of the urine specimen has a low probability of being resistant to these last three antibiotics. The patient X can thus be treated with one of these antibiotics without waiting for the length of time that is necessary for the analysis by antibiogram.

Although the invention has been described in connection with a particular embodiment, it is quite obvious that it is no way limited and that it comprises all of the technical equivalents of means that are described as well as their combinations if the latter fall within the scope of the invention.

1. Process for determination of microorganisms' projected resistance to antibiotics that consists in analyzing a first microorganism by flight and desorption time/matrix-assisted laser ionization mass spectrometry (MALDI-TOF) so as to obtain a first mass spectrum of said first microorganism, whereby said process is characterized in that an analytical device carries out the following stages:
(i) Comparing said first mass spectrum to mass spectra of microorganisms that are identical or at least similar to said first mass spectrum that are contained in a data base, and whose resistance to given antibiotics has already been analyzed by an antibiogram and recorded in said data base so as to obtain a list of resistances to given antibiotics for each identical or similar microorganism,
(ii) Implementing a projected analysis of said first microorganism's resistance to antibiotics based on the list of the resistances to antibiotics obtained in stage (i).

2. Process for determination according to claim 1, wherein the data base is a spectra base that is local to a laboratory and/or a base of predefined spectra, in which the resistance of the organisms associated with these spectra is known.

3. Process for determination according to claim 1, wherein the analytical device comprises a computer and software.

4. Process for determination according to claim 2, wherein the analytical device comprises a computer and software.
